(19) United States
(12) Patent Application Publication
(10) Pub. No.: US 2025/0242089 A1
(43) Pub. Date: Jul. 31, 2025

(54) MEDICAL DEVICE INCLUDING CYCLIC OLEFIN COPOLYMER

(71) Applicant: Covidien LP, Mansfield, MA (US)

(72) Inventors: Edwin Y. Wang, Tustin, CA (US); Xiaojing Guo, Shanghai (CN); Yihan Wang, Shanghai (CN)

(21) Appl. No.: 19/042,702

(22) Filed: Jan. 31, 2025

(30) Foreign Application Priority Data
Jan. 31, 2024 (WO) PCT/CN2024/074988

(51) Int. Cl. A61L 29/04; A61L 29/18

(52) U.S. Cl. CPC A61L 29/049; A61L 29/18

(57) ABSTRACT

A medical device includes an elongated body. At least a portion of the elongated body includes a polymer blend of a polyphthalamide (PPA) and a cyclic olefin copolymer (COC) (PPA/COC). In some medical devices, the COC includes a copolymer of ethylene and norbornene having high stiffness and low moisture uptake, and the PPA includes some amount of renewable carbon content. In some medical devices that include a catheter, a higher stiffness proximal portion of the catheter includes the polymer blend, and a lower stiffness distal portion of the catheter includes an inner liner, an outer jacket, and a structural support member between the inner liner and outer jacket.

### CROSS-REFERENCE TO RELATED APPLICATIONS

This application claims the benefit of and priority to Patent Corporation Treaty Application International Application No. PCT/CN2024/074988 filed Jan. 31, 2024, the entire disclosure of which is incorporated by reference herein.

### TECHNICAL FIELD

This disclosure relates to medical devices, such as catheters and catheter hubs.

### BACKGROUND

Medical devices, such as a medical catheter defining at least one lumen, have been proposed for use with various medical procedures. For example, in some cases, a medical catheter may be used to access and treat defects in blood vessels, such as, but not limited to, lesions or occlusions in blood vessels.

### SUMMARY

This disclosure describes medical devices, such as catheters or catheter hubs, that include an elongated body having improved mechanical performance. At least a portion of the elongated body, such as a proximal portion, includes a blend of a polyphthalamide (PPA) and a cyclic olefin copolymer (COC) (PPA/COC). The COC may have a relatively high stiffness and low moisture uptake compared to other polymers used for medical devices, thereby providing the elongated body with improved pushability, torqueability, and dimensional stability, and in some examples, replacing a structural support member that may otherwise be present. The PPA may include a high content of renewable carbon (e.g., up to about 70 weight percent (wt. %)), thereby reducing an energy and/or material load for manufacturing the elongated body. The portion of the elongated body may be bonded to other portions, such as a distal portion (e.g., a distalmost tip), that may be configured for other properties, such as higher flexibility. For example, these other portions of the elongated body may include a biodegradable polymer outer jacket that can be loaded with radiopaque materials to replace or supplement a marker band. In these various ways, medical devices described herein may have simple construction, adequate mechanical performance, and low environmental load compared to other catheters that do not include a PPA/COC blend.

This disclosure also describes examples of methods of forming the medical devices described herein and methods of using the medical devices.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1 is a conceptual side view of an example catheter, which includes an elongated body at least partially formed from a blend of PPA/COC.

FIG. 2 is a conceptual cross-sectional view of a proximal portion of the elongated body of FIG. 1, where the cross-section is taken along line A-A in FIG. 1 and in a direction orthogonal to a longitudinal axis of the elongated body.

FIG. 3 is a conceptual cross-sectional view of a distal portion of the elongated body of FIG. 1, where the cross-section is taken along line B-B in FIG. 1 and in a direction orthogonal to a longitudinal axis of the elongated body.

FIG. 4 is a conceptual cross-sectional view of an interface between a proximal portion and a distal portion of the elongated body of FIG. 1, where the cross-section is taken at window C in FIG. 1 and in a direction parallel to a longitudinal axis of the elongated body.

FIG. 5 is a conceptual cross-sectional view of a distal portion and distal tip of the elongated body of FIG. 1, where the cross-section is taken at window D in FIG. 1 and in a direction parallel to a longitudinal axis of the elongated body.

FIG. 6 is a flowchart of an example technique for forming an example elongated body of medical devices described herein.

FIG. 7A is a conceptual side view of a portion of an example elongated body, which is formed from a blend of PPA/COC.

FIG. 7B is a graph of a ratio of PPA/COC along a length of the example elongated body of FIG. 7A.

FIG. 7C is a graph of stiffness along a length of the example elongated body of FIG. 7A.

### DETAILED DESCRIPTION

This disclosure describes medical devices, such as a medical catheter or a hub attaching to a medical catheter, that includes a flexible elongated body configured to be navigated through vasculature of a patient. Some typical catheters may be constructed from various functional components, such as an inner liner, an outer jacket, or a structural support member between the inner liner and the outer jacket, that provide particular properties to the catheter. For example, the inner liner may primarily provide lubricity to an inner surface of the catheter, the structural support member may primarily provide stiffness and strength to the catheter, and the outer jacket may primarily provide flexibility to the catheter. However, such functional differentiation may require relatively complex and precise construction techniques, and may lead to potential separation of the components of the catheter. For example, repeated bending of the catheter may cause the relatively flexible outer jacket to delaminate from the relatively stiff structural support member.

At least a portion of the elongated body of medical devices described herein includes a polymer blend of a polyphthalamide (PPA) and a cyclic olefin copolymer (COC) (PPA/COC). The polymer blend provides the portion of the elongated body with sufficient strength and flexibility for navigating the elongated body through vasculature of a patient, and may substitute for both the outer jacket and the structural support member of the elongated body. Both COC and PPA have high stiffness and high strength. The COC has low moisture uptake compared to other polymers used for outer jackets of medical devices. The PPA can be formed from material sources that include a high content of renewable carbon (e.g., up to about 70 weight percent (wt. %)). The proportion of the COC and PPA in the polymer blend may be selected to provide a desired combination of properties for the particular portion of the elongated body, such as a higher proportion of the PPA for stiffer portions of the elongated body and a lower proportion of the PPA for more flexible portions of the elongated body. As a result of the combination of these properties, the polymer blend provides the portion of the elongated body with pushability, torqueability, and dimensional stability, and may reduce an energy and/or material load for manufacturing the portion of the elongated body.

In some examples, a proximal section of the elongated body includes the PPA/COC blend, while the distal section of the elongated body includes a different material or combination of materials that together have a lower stiffness than the proximal section. The lower stiffness distal portion may facilitate navigation of the elongated body through vasculature, as well provide a relatively atraumatic distalmost portion of the elongated body. The proximal section may be coupled to the distal section with a bio-based adhesive material, such as a copolyimide or maleic anhydride. The distal section of the elongated body may include a multiple component configuration (e.g., a structural support member, inner liner, and outer jacket) that is at least partially formed from bio-based materials. For example, the structural support member may include a bio-based fiber, such as bamboo or silk, and/or the outer jacket may include a biodegradable polybutylene adipate terephthalate (PBAT) copolymer that may be loaded with a radiopaque material to form a marker band. In these various ways, the medical devices may have simple construction, adequate mechanical performance, and low environmental load compared to other catheters that do not include a PPA/COC blend.

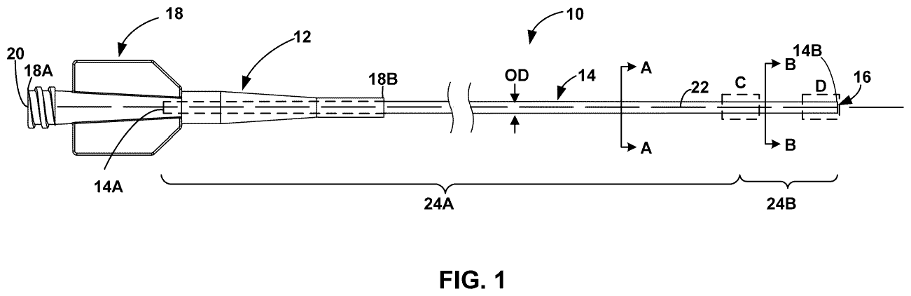

FIG. 1 is a conceptual side view of an example catheter 10, which includes an elongated body 14 at least partially formed from a blend of PPA/COC. Catheter 10 further includes a hub 18 positioned at a proximal end 14A of elongated body 14. Elongated body 14 extends from proximal end 14A to distal end 14B, and defines a proximal portion 24A and distal portion 24B. However, proximal portion 24A and/or distal portion 24B may include further sections, such as a proximal and a medial section of proximal portion 24A and/or a distal section and distal tip of distal portion 24B.

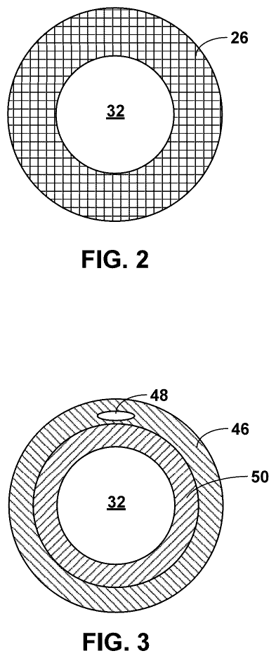

Elongated body 14 may define at least one inner lumen 32 (shown in FIG. 2) that extends the length of elongated body 14. In the example shown in FIG. 1, proximal end 14A of elongated body 14 is received within hub 18 and can be mechanically connected to hub 18 via an adhesive, welding, or another suitable technique or combination of techniques. Opening 20 defined by hub 18 and located at proximal end 18A of hub 18 is aligned with the inner lumen 32 (shown in FIG. 2) of elongated body 14, such that inner lumen 32 of elongated body 14 may be accessed via opening 20. In some examples, catheter 10 may include a strain relief body 12, which may be a part of hub 18 or may be separate from hub 18. In other examples, the proximal end of catheter 10 can include another structure in addition to or instead of hub 18. In some examples, catheter hub 18 may include one or more Luer connectors or other mechanisms for establishing connections between catheter 10 and other devices.

Catheter 10 can be configured for use with any suitable medical procedure. In some examples, inner lumen 32 of elongated body 14 is configured to receive or deliver one or more medical devices, therapeutic agents, etc., to a distal tissue site, remove a thrombus (e.g., by aspiration) from the patient's vasculature, or the like. In examples in which inner lumen 32 is used to remove a thrombus from vasculature, catheter 10 may be referred to as an aspiration catheter. A suction force (e.g., a vacuum) may be applied to a proximal end of catheter 10 (e.g., opening 20) to draw a thrombus into the inner lumen 32. Accordingly, an aspiration system can comprise an aspiration pump connected to the proximal end of catheter 10 (either directly or via intervening tubing or other component(s)).

A clinician may advance catheter 10 to a target location within vasculature of the patient in cooperation with a guide member (not shown) such as a guidewire, an inner catheter, both a guidewire and an inner catheter, or the like, which may aid in the navigation (e.g., steering and manipulation) or elongated body 14 through the vasculature. For example, inner lumen 32 of elongated body 14 may be configured to receive a guide member or an inner catheter, such that the catheter body may be guided through vasculature over the guide member or the inner catheter.

In some examples, elongated body 14 may be used to access relatively distal vasculature locations in a patient, such as the middle cerebral artery (MCA) in a brain of a patient. The MCA, as well as other vasculature in the brain or other relatively distal tissue sites (e.g., relative to the vasculature access point), may be relatively difficult to reach with a catheter, due at least in part to the tortuous pathway (e.g., comprising relatively sharp twists and/or turns) through the vasculature to reach these tissue sites. Elongated body 14 that includes a polymer blend of COC and PPA, alone or in combination with other bio-based materials, may facilitate access to the relatively distal tissue sites by configuring elongated body 14 to be pushable and flexible. For example, forming proximal portion 24A from the polymer blend may strengthen proximal portion 24A and improve pushability of elongated body 14 while enabling elongated body 14 to be relatively flexible to aid navigation to relative distal tissue sites.

In some examples, elongated body 14 may define a consistent outer diameter (OD) or an outer diameter taper (e.g., gradient, gradation, segmented gradient of gradation, or the like). The outer diameter taper may assist with the navigability and/or maneuverability of elongated body 14 through the vasculature of a patient. In some examples, the outer diameter taper may define a continuous transition gradient from an outer diameter of elongated body 14 defined at hub distal end 18B by the outer diameter at distal end 14B of elongated body 14. In other examples, the outer diameter of elongated body 14 may define a discontinuous transition (e.g., a gradation or discrete step-downs) in outer diameter may define a discontinuous transition (e.g., a gradation or discrete step-downs) in outer diameter to define the outer diameter taper.

In some examples, inclusion of the polymer blend of PPA/COC may permit exclusion of a structural support member, thereby enabling the outer diameter of elongated body 14 to remain relatively small to facilitate distal flexibility while still retaining sufficient strength and pushability at proximal portion 24A. Thus, in some examples, proximal portion 24A of elongated body 14 or all of elongated body 14 does not include a structural support member. For example, proximal portion 24A may include COC for pushability, while distal portion 24B may include a structural support member that includes bio-based materials, such as bamboo or silk fibers, coiled around an inner liner and within an outer jacket (not shown in FIG. 1) to provide increased flexibility at distal portion 24B. The increased flexibility may provide catheter 10 with improve navigability and maneuverability. In some examples, distal portion 24B may also be free of a structural support member. For example, distal portion 24B may include a blend of PPA and COC that is relatively soft.

In some examples, the working length of elongated body 14 may be measured from hub distal end 18B of hub 18 (marked by the distal end of optional strain relief body 12) to distal end 14B of distal portion 24C. The working length of catheter 10 may depend on the location of the target tissue and/or the medical procedure for which catheter 10 is used. For examples, if catheter 10 is a distal access catheter used to access vasculature in a brain of a patient from a femoral artery access point at the groin of the patient, catheter 10 may have a working length of about 129 centimeters (cm) to about 135 cm, such as about 132 cm, although other lengths may be used. In other examples, or for other applications, the working length of elongated body 14 may have different lengths.

Proximal and distal portions 24A-24B of elongated body 14 are respective longitudinal sections of elongated body 14. In some examples, combined lengths of proximal and distal portions 24A-24B of elongated body 14 is equal to the working length of elongated body 14. Proximal and distal portions 24A-24B of elongated body 14 may each have any suitable length measured along longitudinal axis 22 of elongated body 14, including the same or different lengths. In some examples, proximal portion 24A has a length of about 70 cm to about 140 cm, such as about 100 cm; and distal portion 24B has a length of about 1 cm to about 25 cm, such as about 2.5 cm to about 10 cm, or about 2.5 cm to about 5 cm. The term “about” as used herein with dimensions may refer to a range within the numerical value resulting from manufacturing tolerances and/or within 1%, 5%, or 10% of the numerical value. For example, a length of about 10 mm refers to a length of 10 mm to the extent permitted by manufacturing tolerances, or a length of 10 mm +/−0.1 mm, +/−0.5 mm, or +/−1 mm in various examples.

In some examples, at least a portion of a surface, such as an inner surface and/or an outer surface, of elongated body 14 includes one or more coatings, such as, but not limited to, an anti-thrombogenic coating, which may help reduce the formation of thrombi in vitro, an anti-microbial coating, and/or a lubricating coating. The lubricating coating can be, for example, a hydrophilic coating. In some examples, at least a portion of an inner surface of elongated body 14, including the portion that includes the polymer blend of PPA/COC, includes a lubricious coating.

While FIG. 1 has been described with respect to a portion of catheter 10 formed from the PPA/COC polymer blend, in some examples, hub 18 may be formed from the polymer blend of PPA/COC.

FIG. 2 is a conceptual cross-sectional view of proximal portion 24A of elongated body 14 of FIG. 1, where the cross-section is taken along line A-A in FIG. 1 and in a direction orthogonal to a longitudinal axis of the elongated body. In the example of FIG. 2, proximal portion 24A of elongated body 14 includes tubular body 26. Tubular body 26 may have any desired thickness suitable for maintaining an inner diameter of inner lumen 32 and an outer diameter of elongated body 14, as well as providing mechanical properties related to pushability of elongated body 14. For example, a thickness of a wall of tubular body 26 may be from about 25 microns to about 200 microns, such as from about 50 microns to about 100 microns.

Tubular body 26 includes a polymer blend of a polyphthalamide (PPA) and a cyclic olefin copolymer (COC) (PPA/COC). A polymer blend may include a mixture of PPA and COC in a relative proportion. Selection of the composition of the PPA, the composition of the COC, and the relative proportion (or ratio) of PPA and COC may determine the various properties of the polymer blend. In general, COCs and PPAs have a variety of properties suitable for use in a medical device inserted into vasculature of a patient. For example, elongated body 14 may be capable of navigating through the vasculature and interacting with tissues and fluids within the vasculature. While not being limited to any particular theory, in some examples, the polymer blend of COC and PPA forms distinct phases within the polymer blend. For example, the polymer blend of COC and PPA may have relatively uniform properties, but may have a microstructure that includes distinct phases of PPA and COC.

COC is an amorphous thermoplastic that is copolymerized from a cyclic olefin (i.e., unsaturated hydrocarbon) polymer, such as norbornene, and a linear olefin polymer, such as ethylene. COCs may include properties favorable to an aqueous environment, such as low water absorption (e.g., less than about 0.01%, per ISO 62), low water vapor permeability (e.g., less than about 0.05 g·mm/m²·d, per DIN 53 122), and high compatibility with blood and tissues. COCs may also include properties favorable for navigation through a tortuous environment, such as low tensile modulus (e.g., less than about 3200 MPa, per ISO 527), low elongation at break (e.g., less than about 2.7%, per ISO 527), high tensile strength (e.g., greater than about 45 MPa per ISO 527), and high hardness (e.g., greater than about 180 N/mm², per ISO 2039). In addition to properties related to performance, COCs may also include properties favorable for manufacturability, such as high thermal stability (e.g., up to about 170° C., per ISO 75), good flowability (e.g., volume flow index greater than about 20 ml/10 min, per ISO 1133), and high stability (e.g., mold shrinkage less than about 0.7%). Particular properties of the COC may be further configured based on a composition and relative proportion of the cyclic polymer and olefin polymer used to form the COC particular copolymer of the COC. For example, a COC formed from a higher proportion of cyclic olefin may have enhanced thermal properties compared to a COC having a lower proportion of cyclic olefin, while a COC formed from a higher proportion of linear olefin may have a higher flowability than a COC having a lower proportion of linear olefin. In some examples, the COC includes a copolymer of ethylene and norbornene.

PPA is a polymer that includes a combination of at least 50 wt. % terephthalic and/or isophthalic acid with one or more diamines. PPAs may include properties favorable to an aqueous environment, such as low water absorption and high resistance to chemicals. In addition to providing advantageous mechanical, thermal, and chemical properties, PPA may be sourced from bio-based feedstocks having a high renewable carbon content. For example, terephthalic acids, isophthalic acids, and diamine monomers or polymers used to form the PPA may be generated from bio-based feedstocks such as vegetable oils, as measured in renewable carbon content (e.g., per ASTM 6866-12). As a result, the PPA/COC blend may include a high amount of renewable carbon content. In some examples, the PPA/COC blend includes greater than or equal to 40 percent by weight renewable carbon content. In some examples, the PPA/COC blend includes less than or equal to about 70 percent by weight renewable carbon content. In some examples, the PPA may include Rilsan HT™ available from Arkema (Serquigny, France).

COCs may be compatible with other thermoplastics, including PPA. For example, COC and PPA are both thermoplastics that can be configured to have relatively similar glass transition temperatures (e.g., a range from about 65° C. to about 180° C. for COC depending on a relative proportion of cyclic olefin, and about 135° C. for PPA). While both COC and PPA have high resistance to moisture uptake and high dimensional stability, COC and PPA may have different stiffnesses, as PPA is stiffer than COC. For example, PPA may have a tensile modulus of about 3.7 GPa, while COC may have a tensile modulus of about 2.6 GPa. As a result, the stiffness of the polymer blend, and correspondingly tubular body 26, may be configured based on the relative proportion of PPA to COC.

In some examples, proximal portion 24A of elongated body 14 is monolithic, such that a macrostructure of tubular body 26 along and across longitudinal axis 22 is uniform. For example, tubular body 26 may be formed from a tube that includes only the PPA/COC blend. As a result, proximal portion 24A of elongated body 14 may not include a structural support member. However, due to the presence and proportion of PPA and COC in the PPA/COC blend, tubular member 26 may have sufficient stiffness to provide pushability without the additional support of a structural support member.

In some examples, proximal portion 24A of elongated body 14 is homogeneous along longitudinal axis 22. In such examples, a proportion of PPA to COC in the polymer blend may be relatively uniform along longitudinal axis 22 of elongated body 14, such that, for a particular thickness of tubular body 26, proximal portion 24A may have a uniform stiffness. While proximal portion 24A is illustrated as being a single section, in other examples, proximal portion 24A may include more than one section, each having a homogeneous composition and different stiffness than other sections. For example, proximal portion 24A may include a first section having a PPA/COC blend with a higher relative proportion of PPA and a second, more distal section having a PPA/COC blend with a lower relative proportion of PPA.

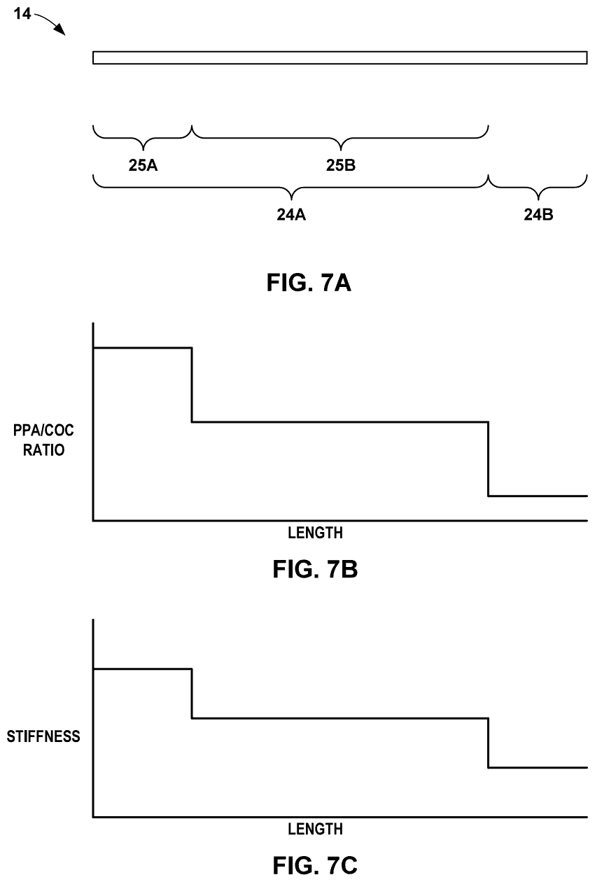

In some examples, proximal portion 24A of elongated body 14 is not homogeneous along longitudinal axis 22. In such examples, a proportion of PPA to COC in the polymer blend may be variable along longitudinal axis 22 of elongated body 14, such that, for a particular thickness of tubular body 26, proximal portion 24A may have a variable stiffness. For example, as illustrated in FIGS. 7A-7C below, a relative proportion of PPA to COC may decrease along longitudinal axis 22, in a step function and/or gradually, such that proximal portion 24A may be stiffer near a connection to hub 18 than at an interface with distal portion 24B. A proportion of PPA to COC in tubular body 26 may be from about 9:1 (e.g., relatively stiff) to about 1:9 (e.g., relatively soft).

FIG. 3 is a conceptual cross-sectional view of distal portion 24B of elongated body 14 of FIG. 1, where the cross-section is taken along line B-B in FIG. 1 and in a direction orthogonal to longitudinal axis 22 of elongated body 14. As shown in FIG. 3, in some examples, distal portion 24B of elongated body 14 includes outer jacket 46, structural support member 48, and inner liner 50, which defines inner lumen 32. Structural support member 48 is positioned between outer jacket 46 and inner liner 50. Structural support member 48 may look different in cross-section in other examples, depending on whether structural support member 48 includes one or more axially extending structures (e.g., a hypotube), coils, or braids. Outer jacket 46, structural support member 48, and/or inner liner 50 may be laminated together to form a single component using any suitable method (e.g., through the application of heat or a soft or flexible adhesive (e.g., polyurethane (PU)).

Structural support member 48 is located between outer jacket 46 and inner liner 50 and provides structural integrity to elongated body 14. For example, structural support member 48 can be configured to improve kink resistance, axial column strength, and/or burst strength of elongated body 14. Outer jacket 46 may protect and contain the other components of elongated body 14 of catheter 10 such as structural support member 48 and inner liner 50. Outer jacket 46 may act as a barrier to help prevent liquids such as a patient's body fluids from entering elongated body 14 and to prevent liquids in inner lumen 32 from exiting elongated body 14. In some examples, outer jacket 46 may be a single, continuous component or may be the result of the longitudinal joining and/or lamination of multiple components. Inner liner 50 defines inner lumen 32 and may be formed from any suitable material. Inner lumen 32 may be sized to receive a medical device (e.g., another catheter, a guidewire, an embolic protection device, a stent, a thrombectomy device, an intrasaccular implant such as a coil, or any combination thereof), a therapeutic agent, or the like. At least the inner surface of inner liner 50 defining inner lumen 32 may be lubricious in some examples in order to facilitate the introduction and passage of a device, a therapeutic agent, or the like, through inner lumen 32.

As described above, a portion of elongated body 14 includes a polymer blend of PPA/COC, in which the PPA may include at least some amount of renewable carbon content. Other portions of elongated body 14, such as distal portion 24B, may also include bio-based and/or biodegradable materials that incorporate renewable materials or reduce use of non-renewable materials. For example, one or more of outer jacket 46, structural support member 48, and/or inner liner 50 may include bio-based and/or biodegradable materials, such that an energy or environmental load associated with manufacture of catheter 10 may be further reduced.

In some examples, structural support member 48 includes a bio-based fiber. A bio-based fiber may include any fiber derived from a bio-based feedstock. A variety of bio-based fibers may be used including, but not limited to, bamboo fibers, silk fibers, biopolymer fibers, or the like. In some examples, the bio-based fiber may be a biodegradable material (e.g., bamboo fibers alone or in combination with silk fibers and/or biodegradable polymer fibers). Structural support member 48 may be formed primarily or entirely of biodegradable materials including bamboo fibers alone, or bamboo fibers in addition to one or more of silk fibers, and/or bioinert fibers beta-calcium meta phosphate, and/or biodegradable polymers fibers (e.g., PLA fibers). For example, bamboo fibers may be located along an entire length of structural support member 48 (a length being measured along longitudinal axis 22) or may be located along only a part of a length of structural support member 48.

In some examples, structural support member 48 is formed from the one or more biodegradable materials throughout elongated body 14, e.g., has the same configuration along an entire length of structural support member 48. For example, structural support member 48 may consist essentially of bamboo fibers (e.g., includes bamboo fibers and other materials that do not materially affect the structural characteristics of structural support member 48. As another example, structural support member 48 may include both bamboo and another biodegradable material, such as silk.

Structural support member 48 may, based on the properties of the biodegradable materials selected, impart different structural properties to elongated body 14. The structural properties include strength, stiffness, pushability, flexibility, navigability, and/or maneuverability of elongated body 14. For example, portions of structural support member 48 comprising bamboo fibers may, based on the properties of bamboo, improve the kink resistance, axial column strength, burst strength, stiffness, and/or pushability of elongated body 14. Portions of structural support member 48 comprising silk fibers and not bamboo fibers may, based on the properties of silk, improve flexibility, navigability, and/or maneuverability of elongated body 14.

Structural support member 48 can include axially extending strengthening elements, a hypotube, a coil, and/or a braid. In some examples, structural support member 48 includes bamboo fibers arranged axially along longitudinal axis 22 and along at least a part (e.g., distal portion 24B) of elongated body 14. In addition to or instead of the axially extending bamboo fibers, structural support member 48 can include silk or bamboo fibers or filaments coiled and/or braided around inner liner 50.

In some examples, outer jacket 46 includes a biodegradable polymer configured to enable outer jacket 46 to decompose over time. A biodegradable polymer may include any polymer that breaks down within a finite period of time to produce natural byproducts. The finite period of time may be longer than the use of the medical device, such as greater than a length of the procedure or treatment, and less than a year. Biodegradable polymers that may be used include, but are not limited to, bio-based polymers, such as polysaccharides and proteins, bio-derived polymers, such as polylactic acid (PLA), and synthetic polymers, such as polycaprolactone and polybutylene adipate terephthalate (PBAT).

In some examples, the biodegradable polymer includes a biodegradable PBAT copolymer. PBAT is a co-polyester that that combines high biodegradability of aliphatic polyesters with high mechanical and thermal properties, such as wear resistance, of aromatic polyesters. Use of PBAT for outer jacket 46 may result in more secure thermal bonding during assembly processes, may reduce misalignment issues, may increase adhesion of distal portion 24B to proximal portion 24A and/or the catheter tip, and may simplify production procedures by dropping typical multi-step reflow processes.

In some examples, the biodegradable polymer includes one or more biodegradable thermoplastics and/or one or more biodegradable thermoset polymers (e.g., PLA, PGLA, and/or PBAT). In some examples in which outer jacket 46 is formed primarily or entirely of a PLA, PBAT, and/or PGLA fiber, outer jacket 46 may be configured to decompose when exposed to an enzyme that degrades the PLA PBAT, and PGLA fiber into innocuous lactic acid, carbon dioxide, and water matrix. This decomposition may occur from, for example, a time from of 6 months to 2 years. The biodegradation can occur, for example, by diffusion of body fluid, enzyme and water, between the polymer chains. In some examples in which other parts of elongated body 14 are formed from a biodegradable material, such as bamboo and/or silk, these other parts of elongated body 14 can also be configured to degrade in the same or similar time frame and in response to the same one or more enzymes.

In some examples, inner liner 50 may also comprise a biodegradable polymer (e.g., biodegradable thermoplastics and/or biodegradable thermoset polymers), which can be the same biodegradable polymer or different biodegradable polymers than outer jacket 46. In some examples, inner liner 50 is formed primarily or entirely of biodegradable polymers, such as, biodegradable thermoplastics and/or biodegradable thermoset polymers. In other, inner liner 50 may comprise polytetrafluoroethylene (PTFE). Although one inner lumen 32 is shown in FIGS. 2 and 3, in other examples, elongated body 14 can define multiple inner lumens.

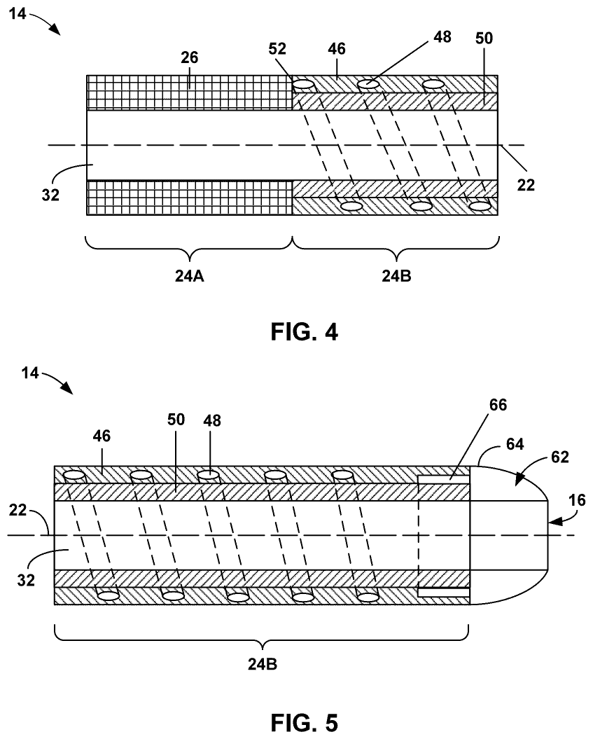

FIG. 4 is a conceptual cross-sectional view of an interface between proximal portion 24A and distal portion 24B of elongated body 14 of FIG. 1, where the cross-section is taken at window C in FIG. 1 and in a direction parallel to longitudinal axis 22 of elongated body 14. A distal-most end of proximal portion 24A interfaces with a proximal-most end of distal portion 24B, either directly or through one or more layers (e.g., an adhesive layer). In some examples, elongated body 14 further includes a bio-based adhesive material at a junction 52 between proximal portion 24A and distal portion 24B. For example, the bio-based adhesive material may include a copolyimide or a maleic anhydride-based material.

FIG. 5 is a conceptual cross-sectional view of distal portion 24B and a distal tip of elongated body 14 of FIG. 1, where the cross-section is taken at window D in FIG. 1 and in a direction parallel to longitudinal axis 22 of elongated body 14. The cross-sectional view of FIG. 5 illustrates outer jacket 46, structural support member 48, inner liner 50, and inner lumen 32. Structural support member 48 is shown as including a bio-based fiber as a coil, but structural support member 48 can include a bio-based braided structure and/or axially extending fibers, alone or in combination with the coil. While the cross-sectional view of FIG. 5 includes distal tip member 62, other examples of catheter 10 may not include distal tip member 62 and/or a polymeric radiopaque marker 66.

Distal tip member 62 defines an atraumatic tip of catheter 10. In the example shown in FIG. 5, distal tip member 62 is at a distal-most end of elongated body 14, and defines distal-most opening 16 to inner lumen 32. In some examples, distal tip member 62 includes a hydrophilic coating 64, which may reduce surface friction and enhance lubricity of distal tip member 62. In some examples, distal tip member 62 is formed from one or more biodegradable polymers, such as one or more biodegradable thermoplastics and/or biodegradable thermoset polymers (e.g., PBAT and/or PTFE). Distal tip member 62 may be detachable from distal portion 24B without compromising the structural integrity of elongated body 14.

In some examples, elongated body 14 includes a radiopaque marker 66 to facilitate fluoroscopic or MRI visualization of a distal portion 24B of elongated body 14. Radiopaque marker 66 may form a ring or partial ring that extends around the outer perimeter of distal tip member 62, inner liner 14, or distal portion 24B of elongated body 14. In some examples, radiopaque marker 66 may be laminated with inner liner 50, outer jacket 46, and/or other parts of distal portion 24B of elongated body 14.

Rather than form radiopaque marker 66 from a solid metallic material, radiopaque marker 66 may include one or more biodegradable polymers (e.g., PBAT and/or PTFE) and one or more radiopaque materials (e.g., barium sulfate, tantalum, tungsten, platinum, and/or other relatively high-density radiopaque materials) loaded into the one or more biodegradable polymers. For example, distal portion 24B of outer jacket 46 may include a PBAT copolymer filled with a radiopaque material, such as at least one of tungsten, bismuth oxychloride, or bismuth trioxide. A metallic radiopaque marker (e.g., a platinum band) may contribute to the overall stiffness of a distal tip of a catheter, as well as render the catheter unsuitable for use with MRI. Forming marker band 66 from a radiopaque polymer (a polymer including a radiopaque material distributed therein) may enable the metallic radiopaque marker to be eliminated from the distal tip, thereby enabling the distal portion of catheter 10 to be more flexible. In addition, enabling the metallic radiopaque marker band to be eliminated from catheter 10 without compromising the visibility of catheter 10 under medical imaging may enable distal portion 24B of elongated body 14 and/or the entire distal portion 24B of elongated body 14 to be formed primarily or entirely of biodegradable materials.

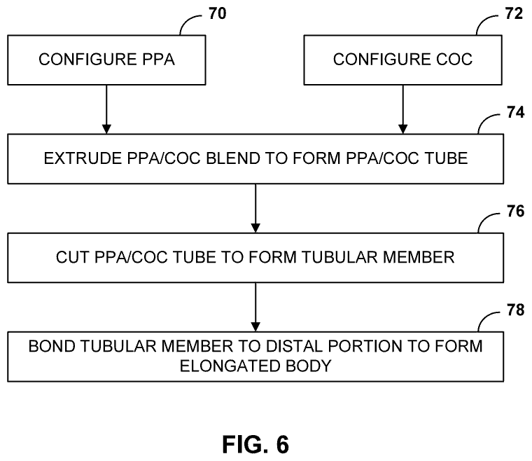

FIG. 6 is a flowchart of a technique for forming an example elongated body of medical devices described herein. The technique of FIG. 6 includes configuring PPA (70). Configuring the PPA may include selecting a composition of PPA for various properties, such as flowability, glass transition temperature, or renewable carbon content. The method of FIG. 6 also includes configuring COC (72). Configuring the COC may include selecting a composition of COC for various properties, such as flowability, glass transition temperature, or stiffness.

The technique of FIG. 6 includes extruding the PPA and COC to form a PPA/COC tube (74). Prior to or during extrusion, the PPA and COC may be selected in relative properties for a particular stiffness or stiffness profile (e.g., stiffness along a longitudinal axis). The PPA and COC may be separately melted and blended during extrusion, thereby providing a particular relative proportion of the PPA and the COC in the particular PPA/COC blend. For example, the PPA and COC may be mixed in particulate or pelletized form prior to melting. Such particular relative proportion of PPA and COC may be varied as the polymer blend is extruded along an axis of the tube, thereby providing a varying stiffness along the length of the tube. The technique of FIG. 6 includes cutting the PPA/COC tube to form a tubular member, such as tubular member 26 of FIG. 2 (76). The PPA/COC tube may be cut to a length corresponding to proximal portion 24A of elongated body 14 of FIG. 1.

The technique of FIG. 6 further includes bonding tubular member 26 to a distal portion, such as distal portion 24B, to form elongated body 14. As one example, a bio-based adhesive may be applied to a junction between tubular member 26 and distal portion 24B to bond proximal portion 24A to distal portion 24B. As another example, tubular member 26 and one or more portions of distal portion 24B, such as outer jacket 46, may be reflowed and cooled to bond tubular member 26 to distal portion 24B. In this way, medical devices described herein may be formed from relatively simple extrusion and/or cutting techniques.

FIG. 7A is a conceptual side view of a portion of an example elongated body, which is formed from a blend of PPA/COC. In the example of FIG. 7A, elongated boy 14 includes a proximal portion 24A and a distal portion 24B, in which the proximal portion 24A further includes a distal section 25A and a medial section 25B. Distal section 25A may be configured to couple to a lure hub or other conduit for connecting elongated body 14 to a treatment system or device. Medial section 25B may be configured to transmit a pushing force from the relatively stiff proximal section 25A to the relatively soft distal portion 24B. Distal portion 24B, as described above, may be configured to navigate to a treatment site.

In the example of FIG. 7A, elongated body 14 may be formed as a continuous, in that distal section 25A, medial section 25B, and distal portion 24B may be either unitary or bonded together. While each section of elongated body 14 may include COC and PPA, the blend of PPA and COC may vary in each section. FIG. 7B is a graph of a ratio of PPA/COC along a length of the example elongated body of FIG. 7A. As illustrated in FIG. 7B, distal section 25A may have a relatively high ratio of PPA to COC, such as from pure PPA to about 6:3 PPA to COC (e.g., about 9:1 PPA to COC); medial section 25B may have a relatively moderate ratio of PPA to COC, such as about 6:3 PPA to COC to about 3:6 PPA to COC (e.g., about 5:4 PPA to COC); and distal portion 24B may have a relatively low ratio of PPA to COC, such as from about 3:6 PPA to COC to pure COC (e.g., about 1:9 PPA to COC).

FIG. 7C is a graph of stiffness along a length of the example elongated body of FIG. 7A. As illustrated in FIG. 7C, proximal section 25A having a relatively high ratio of PPA to COC has a relatively high stiffness; medial section 25B having a relatively moderate ratio of PPA to COC has a relatively moderate stiffness; and distal portion 24B having a relatively low ratio of PPA to COC has a relatively low stiffness. While FIG. 7C is illustrated as including step functions in stiffness, a change in stiffness may be more gradual.

The examples described herein may be combined in any permutation or combination.

Example 1: A medical device includes an elongated body, wherein at least a portion of the elongated body comprises a blend of a polyphthalamide (PPA) and a cyclic olefin copolymer (COC) (PPA/COC).

Example 2: The medical device of example 1, wherein the COC comprises a copolymer of ethylene and norbornene.

Example 3: The medical device of any of examples 1 and 2, wherein the PPA/COC blend includes greater than or equal to 40 percent by weight renewable carbon content.

Example 4: The medical device of any of examples 2 and 3, wherein the PPA/COC blend includes less than or equal to about 70 percent by weight renewable carbon content.

Example 5: The medical device of any of examples 1 through 4, wherein the portion of the elongated body is monolithic and homogeneous.

Example 6: The medical device of any of examples 1 through 5, wherein the elongated body comprises a proximal portion and a distal portion, and wherein at least the proximal portion comprises the PPA/COC blend.

Example 7: The medical device of example 6, wherein the distal portion of the elongated body comprises a material different from the PPA/COC blend.

Example 8: The medical device of example 7, wherein a stiffness of the material of the distal portion is less than a stiffness of the PPA/COC blend.

Example 9: The medical device of any of examples 7 and 8, wherein the elongated body further comprises a bio-based adhesive material at a junction between the proximal portion and the distal portion.

Example 10: The medical device of example 9, wherein the bio-based adhesive material comprises a copolyimide or a maleic anhydride-based material.

Example 11: The medical device of any of examples 6 through 10, wherein the elongated body comprises a catheter.

Example 12: The medical device of example 11, wherein the distal portion of the elongated body comprises: an inner liner; a structural support member; and an outer jacket, wherein the structural support member is positioned between the inner liner and the outer jacket.

Example 13: The medical device of example 12, wherein the portion of the elongated body does not include a structural support member.

Example 14: The medical device of any of examples 12 and 13, wherein the outer jacket comprises a biodegradable polybutylene adipate terephthalate (PBAT) copolymer.

Example 15: The medical device of example 14, wherein a distal portion of the outer jacket comprises the PBAT copolymer filled with a radiopaque material.

Example 16: The medical device of example 15, wherein the radiopaque material comprises at least one of tungsten, bismuth oxychloride, or bismuth trioxide.

Example 17: The medical device of any of examples 12 through 16, wherein the structural support member comprises a bio-based fiber.

Example 18: The medical device of example 17, wherein the bio-based fiber comprises at last one of bamboo or silk.

Example 19: The medical device of any of examples 1 through 18, wherein an inner surface of the portion of the elongated body comprises a lubricious coating.

Example 20: The medical device of any of examples 1 through 19, wherein the elongated body comprises a portion of a catheter hub.

Various aspects of the disclosure have been described. These and other aspects are within the scope of the following claims.

What is claimed is:

1. A medical device comprising:
an elongated body, wherein at least a portion of the elongated body comprises a blend of a polyphthalamide (PPA) and a cyclic olefin copolymer (COC) (PPA/COC).

2. The medical device of claim 1, wherein the COC comprises a copolymer of ethylene and norbornene.

3. The medical device of claim 1, wherein the PPA/COC blend includes greater than or equal to 40 percent by weight renewable carbon content.

4. The medical device of claim 1, wherein the PPA/COC blend includes less than or equal to about 70 percent by weight renewable carbon content.

5. The medical device of claim 1, wherein the portion of the elongated body is monolithic and homogeneous.

6. The medical device of claim 1,
wherein the elongated body comprises a proximal portion and a distal portion, and
wherein at least the proximal portion comprises the PPA/COC blend.

7. The medical device of claim 6, wherein the distal portion of the elongated body comprises a material different from the PPA/COC blend.

8. The medical device of claim 7, wherein a stiffness of the material of the distal portion is less than a stiffness of the PPA/COC blend.

9. The medical device of claim 7, wherein the elongated body further comprises a bio-based adhesive material at a junction between the proximal portion and the distal portion.

10. The medical device of claim 9, wherein the bio-based adhesive material comprises a copolyimide or a maleic anhydride-based material.

11. The medical device of claim 6, wherein the elongated body comprises a catheter.

12. The medical device of claim 11, wherein the distal portion of the elongated body comprises:
an inner liner;
a structural support member; and
an outer jacket,
wherein the structural support member is positioned between the inner liner and the outer jacket.

13. The medical device of claim 12, wherein the portion of the elongated body does not include a structural support member.

14. The medical device of claim 12, wherein the outer jacket comprises a biodegradable polybutylene adipate terephthalate (PBAT) copolymer.

15. The medical device of claim 14, wherein a distal portion of the outer jacket comprises the PBAT copolymer filled with a radiopaque material.

16. The medical device of claim 15, wherein the radiopaque material comprises at least one of tungsten, bismuth oxychloride, or bismuth trioxide.

17. The medical device of claim 12, wherein the structural support member comprises a bio-based fiber.

18. The medical device of claim 17, wherein the bio-based fiber comprises at last one of bamboo or silk.

19. The medical device of claim 1, wherein an inner surface of the portion of the elongated body comprises a lubricious coating.

20. The medical device of claim 1, wherein the elongated body comprises a portion of a catheter hub.
